(19) United States
(12) Patent Application Publication
(10) Pub. No.: US 2018/0161459 A1
(43) Pub. Date: Jun. 14, 2018

(54) Methods of Screening for Drugs to Prevent Noise-Induced Hearing Loss

(71) Applicant: Sound Pharmaceuticals Incorporated, Seattle, WA (US)

(72) Inventors: Jonathan Kil, Seattle, WA (US); Eric D. Lynch, Lake Forest Park, WA (US)

(21) Appl. No.: 15/507,738

(22) PCT Filed: Sep. 2, 2015
(86) PCT No.: PCT/US15/48178

§ 371 Date: Feb. 28, 2017

(51) Int. Cl. A61K 49/00; A61B 5/12

(52) U.S. Cl. CPC A61K 49/0004; A61B 2503/42; A61B 5/123; A61B 5/125

(57) ABSTRACT

The invention relates to methods of identifying a composition useful for the prevention or treatment of noise-induced hearing loss. The method includes exposing a mammalian test subject to a candidate composition and a calibrated sound or noise challenge. Next, a temporary threshold shift (TTS) is monitored in the test subject over a period of time. The monitored TTS is compared with a TTS of a control subject exposed to a control composition. The presence or absence is determined by clinically relevant and statistically significant differences between the monitored TTS in the test subject and the TTS of the control subject, and the presence of a statistically significant difference identifies the candidate composition as useful for prevention or treatment of noise-induced hearing loss.

### CROSS REFERENCE TO RELATED APPLICATIONS

This application claims the benefit of U.S. Provisional Application No. 62/043,944, filed Aug. 29, 2014, which is hereby incorporated by reference in its entirety.

### BACKGROUND

### Field of the Invention

The invention relates to methods of identifying compositions useful for the prevention or treatment of noise-induced hearing loss.

### Description of Related Art

Exposure to loud noise is one of the major causes of noise-induced hearing loss (NIHL). Millions of people are regularly exposed to hazardous noise levels, especially individuals working in the fields of construction, manufacturing, forestry and agriculture, mining, the military, and the music business, among other occupations. Presently, there are no effective treatments for acute or chronic NIHL. What has severely impaired the clinical development of an otoprotective drug for NIHL, is the lack of a reliable method for determining, in the acute period, compounds effective in individuals at risk for developing NIHL. NIHL begins as a Noise Induced Temporary Threshold Shift (TTS). Over time, the TTS does not resolve and becomes a Permanent Threshold Shift (PTS) or NIHL. The CDC states that 31 million Americans have hearing loss due to excessive noise exposure. This is a staggering number for which no FDA approved drugs exist. Researchers are currently exploring select compounds as potential drugs to prevent or treat hearing loss (Lynch, E. and Kil, J., Development of Ebselen, a Glutathione Peroxidase Mime, for the Prevention and Treatment of Noise-Induced Hearing Loss, Semin. Hear. 2009, 30: 047-055). However, novel methods need to be developed and validated in humans to appropriately select the most effective compounds for the prevention or treatment of noise-induced hearing loss. Such a method would rapidly determine a clinically relevant and statistically significant reduction in the incidence, severity and duration of the TTS.

### SUMMARY

Disclosed herein are methods of identifying a composition useful for the prevention or treatment of noise-induced hearing loss. The method includes exposing a mammalian test subject to a candidate composition. The test subject is also exposed to a calibrated sound or noise challenge. A temporary auditory threshold shift (TTS) is monitored in the test subject over a period of time after exposing the test subject to the calibrated sound or noise challenge. The monitored TTS is compared with a TTS of a control subject exposed to a control composition. The presence or absence is determined of a clinically relevant and statistically significant difference between the monitored TTS in the test subject and the TTS of the control subject wherein the presence of a clinically relevant and statistically significant difference identifies the candidate composition as useful for prevention or treatment of noise-induced hearing loss.

Also disclosed herein are methods for identifying a subject at risk for noise-induced hearing loss by exposing a mammalian test subject to a calibrated sound or a noise challenge, monitoring a temporary threshold shift (TTS) in the test subject over a period of time after exposing the test subject to the calibrated sound or noise challenge, comparing the monitored TTS with a control TTS, and determining the presence or absence of a clinically relevant and statistically significant difference between the monitored TTS in the test subject and the control TTS, where the presence of a statistically significant difference identifies the test subject as being at risk for noise-induced hearing loss.

In one embodiment, the test subject is exposed to the candidate composition prior to exposing the test subject to the calibrated sound or noise challenge. In another embodiment, the test subject is exposed to the candidate composition after exposing the test subject to the calibrated sound or noise challenge. In some embodiments, the test subject is a human. The test subject can also be a mouse, rat, guinea pig, chinchilla, or monkey. In some embodiments, the candidate composition is either administered orally, transdermally, or transnasally.

In an embodiment, the candidate composition is selected front the group comprising: a glutathione peroxidase mimic, a xanthine oxidase inhibitor, a glutathione, and a glutathione precursor. In another embodiment, the candidate composition is selected from the group comprising: methionine, N-acetyl-DL-methionine, S-adenosylmethionine, cysteine, homocysteine, N-acetylcysteine, glutathione, glutathione ethylester, glutathione diethylester, glutathione triethylester, cysteamine, cystathione, N,N′-diacetyl-L-cystine (DiNAC), 2(R,S)-D-ribo-(1′,2′,3′,4′-tetrahydroxybutyl)-thiazolidirie (RibCyst), 2-oxo-L-thiazolidine-4-carboxylic acid (OTCA), allopurinol, 1-methylallopurinol, 2-methylallopurinol, 5-methylallopurinol, 7-methylallopurinol, 1,5-dimethylallopurinol, 2,2-dimethylallopurinol, 1,7-dimethylallopurinol, 2,7-dimethylallopurinol, 5,7-dimethylallopurinol, 2,5,7-trimethylallopurinol, 1-ethyoxycarbonylallopurinol, 1-ethoxycarbonyl-5-methylallopurinol, 2-phenyl-1,2-benzoisoselenaol-3(2H)-one (ebselen), 6A,6B-diseleninic acid-6A′,6B′-selenium bridged β-cyclodextrin (6-diSeCD), and 2,2′-diseleno-bis-β-cyclodextrin (2-diSeCD). In another embodiment, the candidate composition is co-administered to the patient combined with a xanthine oxidase inhibitor, a glutathione, or a glutathione precursor.

In an embodiment, the test subject is exposed to the calibrated sound or noise challenge for a duration of time between 1 minute and 10 hours. In a further embodiment, the test subject is exposed to the calibrated sound or noise challenge for a duration of time between 0.5 hours and 4 hours. In a further embodiment, the test subject is exposed to the calibrated sound or noise challenge for 4 hours. In one embodiment, the test subject is exposed to the calibrated sound or noise challenge at 80-110 dBA SPL. In a further embodiment, the test subject is exposed to the calibrated sound or noise challenge at 85-100 dBA SPL.

In one embodiment, the TTS is monitored by serial pure tone audiometry, auditory brainstem responses, distortion product otoacoustic emissions, or a speech discrimination test. In a further embodiment, when the TTS is monitored by serial pure tone audiometry, the serial pure tone audiometry and subsequent analyses determines an incidence, severity, or duration of the monitored TTS. In a further embodiment, the speech discrimination test is a Words in Noise Test (WINT). In an embodiment, the period of time during which the TTS is monitored is between 2 minutes and 7 days. In a further embodiment, the period of time is between 15 minutes and 24 hours. In another embodiment, the test subject has normal hearing or slight hearing loss. In one embodiment, the clinically relevant and statistically significant difference is a 20%-60% difference between an incidence of the monitored TTS and an incidence of the TTS of the control subject.

In one embodiment, the candidate composition is administered to the test subject at a dosage between 100 mg and 2000 mg. In a further embodiment, the candidate composition is administered to the test subject at a dosage of 200 mg, 400 mg, or 600 mg. In another embodiment, the candidate composition is administered to the test subject twice daily for four days. In a further embodiment, a first administration of the candidate composition occurs two days before exposing the test subject to the calibrated sound or noise challenge.

In one embodiment, the test subject and the control subject are the same subject. In another embodiment, the test subject comprises a population of test subjects and the control subject comprises a population of control subjects. In an embodiment, the test subject and the control subject are different subjects. In another embodiment, the test subject is between 18 and 31 years in age. In another embodiment, the test subject had passed a pure tone audiometry test prior to exposing the subject to the candidate composition, and prior to exposing the subject to the calibrated sound or noise challenge.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 illustrates subjects having at least a 10 dB shift at 15 minutes post-sound exposure, according to an embodiment.

FIG. 2 illustrates numbers of subjects having a higher incidence of a return to baseline hearing on the day of sound exposure, according to an embodiment.

FIG. 3 illustrates the results of a Words in Noise Test (WINT) for all subbjects, a 2-dB cohort, and a 5-dB cohort, according to an embodiment,

FIG. 4 illustrates the results of a Words in Noise Test (WINT) for all subjects, a 2-dB cohort, and a 5-dB cohort at 0, 4, and 8 dB signal to noise ratio (SNR), according to an embodiment.

FIG. 5 illustrates the results of a Words in Noise Test (WINT) for all subjects, a 2-dB cohort, and a 5-dB cohort at 0 and 4 dB signal to noise ratio (SNR), according to an embodiment.

### DETAILED DESCRIPTION

Embodiments of the invention disclosed herein include methods of identifying a composition useful for the prevention or treatment of noise-induced hearing loss.

### Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

The term “noise-induced hearing loss” refers to a loss of hearing due to noise (a loud sound or unwanted sound). The hearing loss can be temporary or permanent. Harmful levels of noise cause the injury or loss of sensory and non-sensory cells in the cochlea which are crucial for the sense of hearing. Typically, exposure to such harmful levels of noise, either over a short or long period of time, ultimately results in noise-induced hearing loss.

The term “temporary auditory threshold shift” or TTS refers to a temporary increase in the threshold of hearing—a temporary loss of hearing. After exposure to harmful levels or durations of noise, a subject's sensitivity to sound may be reduced for some time after the noise. If a subject is exposed to too much noise without sufficient recovery time, the loss of hearing could become permanent.

The term “candidate composition” refers to any composition or compound. In particular, a candidate composition can be a test compound that is screened as a potentially useful composition in the prevention or treatment of noise-induced hearing loss.

The term “calibrated sound” refers to a sound or noise that has been spectrally analyzed for frequency (Hz) and intensity or pressure (dB).

The term “noise challenge” refers to a loud sound that is delivered in a controlled setting, typically with calibrated earphones or headphones, for a defined duration.

The term “dBA SPL” refers to decibels A-weighted (dBA), and sound pressure level (SPL). The dBA SPL is an A-weighted decibel measure of the effective sound pressure of a sound relative to a standard reference value.

The term “otoprotectant” refers to any therapeutic agent that can prevent or ameliorate hearing loss.

The term “subject” encompasses an organism, human or non-human, male or female. The subject can be a human patient. The term “human” generally refers to Homo sapiens. The term “mammal” as used herein includes but is not limited to a human, non-human primate, mouse, rat, guinea pig, chinchilla and monkey. Mammals other than humans can be advantageously used as subjects that represent animal models of, e.g., hearing loss.

The term “statistically significant” is defined as lime probability that a result is not caused by random chance.

The teem “clinically relevant” is defined as a 10 dB threshold shift or a return to baseline hearing on the day of the noise induced hearing loss, or within 24 hours of the noise induced hearing loss.

It must be noted that, as used in the specification and the appended claims, the singular forms “a,” “an,” and “the” include plural referents unless the context clearly dictates otherwise.

### Methods of the Invention

Methods of identifying compositions that are useful for the prevention or treatment of noise-induced hearing loss are disclosed.

In an embodiment, a mammalian test subject is exposed to a candidate composition. The test subject is generally a human, but can also be a non-human primate, rat, mouse, guinea pig, chinchilla, monkey, or other mammal. The test subject can be any age, for example between 18 and 31 years in age. In some cases, the test subject has normal hearing. In other cases, the test subject has slight hearing loss, for example less than or equal to 25 dB HL.

The candidate composition is any composition or compound. Preferably, the candidate composition is an otoprotectant. In an embodiment, the candidate composition is a glutathione peroxidase mimic. Glutathione peroxidase reduces reactive oxygen species by the binding of free radicals to its Se moiety. By reacting with glutathione, glutathione peroxidase limits free radical toxicity, exhibiting strong activity against peroxynitrite. Ebselen is an example of a glutathione peroxidase. Ebselen is known to reduce cytochrome C release from mitochondria and nuclear damage during lipid peroxidation, attenuating neuronal apoptosis associated with oxidative stress. Agents that reduce the activity of reactive oxygen species can attenuate the deleterious effects of loud sounds or noise.

In other embodiments, the candidate composition is selected from the group including: a glutathione peroxidase mimic, a xanthine oxidase inhibitor, a glutathione, and a glutathione precursor. More specifically, the candidate composition can be selected from the group including: methionine, N-acetyl-DL-methionine, S-adenosylmethionine, cysteine, homocysteine, N-acetylcysteine, glutathione, glutathione ethylester, glutathione diethylester, glutathione triethylester, cysteamine, cystathione, N,N′-diacetyl-L-cystine (DiNAC), 2(R,S)-D-ribo-(1′,2′,3′,4′-tetrahydroxybutyl)-thiazolidine (RibCyst), 2-oxo-L-thiazolidine-4-carboxylic acid (OTCA), allopurinol, 1-methylallopurinol, 2-methylallopurinol, 5-methlallopurinol, 7-methylallopurinol, 1,5-dimethylallopurinol, 2,2-dimethylallopurinol, 1,7-dimethylallopurinol, 2,7-dimethylallopurinol, 5,7-dimethylallopurinol, 2,5,7-trimethylallopurinol, 1-ethyoxycarbonylallopurinol, 1-ethoxycarbonyl-5-methylallopurinol, 2-phenyl-1,2-benzoisoselenaol-3(2H)-one (ebselen), 6A,6B-diseleninic acid-6A′,6B′-selenium bridged β-cyclodextrin (6-diSeCD), and 2,2′-diseleno-bis-β-cyclodextrin (2-diSeCD).

Exposing the candidate composition to the test subject can include administering a therapeutic amount of the candidate composition to the test subject. The candidate composition can be administered to the test subject using any method. For example, the candidate composition can be administered orally, transdermally, transnasally, precutaneously, topically, or by aerosol spray. In one embodiment, the candidate composition is administered to the test subject at a dosage between 100 mg and 2000 mg. For example, the candidate composition can be administered to the test subject at a dosage of 50 mg, 100 mg, 200 mg, 300 mg, 400 mg, 500 mg, 600 mg, 700 mg, 800 mg, 900 mg, 1000 mg, 1500 mg, or 2000 mg. A specified dosage of the candidate composition can be administered to the test subject once a day, twice a day, three times a day, or four or more times a day. Also, the candidate composition can be administered at a dosage of one or more times a day for one day, two days, three days, four days, five days, six days, one week, or more than one week. In one example, the candidate composition is administered to the test subject twice daily for four days. In a further example, the candidate composition is first administered to the subject two days before exposing the test subject to a calibrated sound or noise challenge. However, the candidate composition can be administered to the subject anytime before exposing the test subject to a calibrated sound or noise challenge, for example one day, two days, three days, four days, five days, six days, or one week before. Furthermore, the candidate composition can be co-administered to the test subject combined with another composition, such as a xanthine oxidase inhibitor, a glutathione, a glutathione precursor, or any other compound.

The test subject is also exposed to a calibrated sound or a noise challenge. The exposure to the candidate composition can occur either prior to or after exposing the test subject to the calibrated sound or noise challenge. In some cases, the exposure to the candidate composition and the calibrated sound or noise challenge occurs simultaneously. Furthermore, in an embodiment the test subject had passed a pure tone audiometry test prior to exposing the subject to the candidate composition and the calibrated sound or noise challenge. The calibrated sound or noise challenge can come from a variety of sources, such as music from a digital music player (an iPod®, etc.), via insert earphones.

In some embodiments, the test subject is exposed to the calibrated sound or noise challenge for a duration of time between 1 minute and 10 hours. Preferably, the duration of time is between 0.5 hours and 4 hours. In one example, the duration of time is specifically 4 hours. In an embodiment, the test subject is exposed to the calibrated sound or noise challenge at 80-110 dBA SPL. Preferably, the test subject is exposed to the calibrated sound or noise challenge at 85-100 dBA SPL.

A temporary threshold shift (TTS) is monitored in the test subject over a period of time after exposing the test subject to the calibrated sound or noise challenge. The TTS can be monitored by a variety of techniques, including serial pure tone audiometry, auditory brainstem responses, or distortion product otoacoustic emissions. If the TTS is monitored by serial pure tone audiometry, an incidence, severity, or duration of the monitored TTS can be determined. In some cases, the TTS is monitored over a period of time that lasts between 2 minutes and 7 days. Preferably, the period of time lasts between 15 minutes and 24 hours.

The monitored TTS is compared with a TTS of a control subject exposed to a control composition. In some embodiments, the control composition is a placebo. The placebo can contain, for example, microcrystalline cellulose, croscarmellose sodium, and/or magnesium stearate. The control subject is generally a mammalian subject. The control subject can be a human, a non-human primate, rat, mouse, guinea pig, chinchilla, monkey, or other mammal. The test subject and the control subject can be the same subject, or can be different subjects. In an embodiment, the test subject includes a population of test subjects and the control population includes a population of control subjects.

The presence or absence of a clinically relevant and statistically significant difference between the monitored TTS in the test subject and the TTS of the control subject is determined, and the presence of a clinically relevant and statistically significant difference identifies the candidate composition as useful for the prevention or treatment of noise-induced hearing loss. In an embodiment, a clinically relevant and statistically significant difference is defined as p≤0.01 or p≤0.05. In another embodiment, the clinically relevant and statistically significant difference is anywhere between 20%-60% between an incidence of the monitored TTS and an incidence of the TTS of the control subject.

Methods of the invention can also be used to identify a subject at risk for NIHL. In one example, a TTS is monitored in a test subject who has not necessarily been exposed to a candidate composition. This monitored TTS is compared with a control TTS. The control TTS is representative of a subject who has not been exposed to a calibrated sound or noise challenge. In other words, the control TTS is a baseline TTS. The presence or absence of a statistically significant difference between the monitored TTS and control TTS is determined. If there is a statistically significance difference, the subject can be diagnosed as being at risk for NIHL.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Example 1

### A Glutathione Peroxidase Mimic and Inducer for Prevention and Treatment of Noise-Induced Hearing Loss

A study was performed to evaluate the safety and efficacy of an orally delivered candidate composition in human subjects. The candidate composition ebselen (aka. SPI-1005) was evaluated for efficacy using the methods disclosed herein. Ebselen or placebo was administered in a randomized double-blinded study to subjects, who were then exposed to a calibrated sound or noise challenge. Post-sound exposure pure tone audiometry was compared with baseline (i.e., immediately pre-sound exposure) testing to determine group mean level hearing threshold shift changes between treated and placebo groups. The single center, randomized, double-blinded, placebo-controlled study enrolled 83 subjects, all between the ages of 18 and 31. All enrolled subjects ranged from having slight hearing loss (less than or equal to 25 dB HL) to normal hearing.

Each subject was instructed to avoid non-occupational sound exposure (e.g., concerts, firearms, fireworks, power tools) during the 24-hour period preceding baseline testing and throughout the duration of the study. Vital signs (i.e., heart rate, blood pressure, respirations, temperature) were within normal limits for each subject. Subjects had conventional audiologic assessments at baseline consisting of: (a) pure tone audiometry to confirm that subjects had symmetric hearing with air conduction thresholds no worse than 25 dB HL at frequencies between 0.25 to 8 kHz bilaterally, (b) no significant threshold asymmetry (i.e. greater than 15 dB) between any subject's ears at any tested frequency,(c) no significant air-bone gaps (i.e. greater than 10 dB). Bone conduction testing with masking as conducted at screening at 250, 500, 1000, 2000, or 4000 Hz if the air-conduction threshold was greater than 15 dB HL, and (d) Type A tympanograms bilaterally, defined as a range of −140 to +40 daPa based on the 90% range for adults. Subjects with pure tone audiometry hearing thresholds>25 dB HL at any tested frequency (250, 500, 1000, 2000, 3000, 4000, 6000, and 8000 Hz) for either ear were excluded from the study.

Ebselen, or 2-phenyl-1,2-benzisoselenazol-3 (2H)-one, was selected as a candidate composition. The ebselen formulation was a capsule containing 200 mg of ebselen and 150 mg of the excipients microcrystalline cellulose, sodium croscarmellose, and magnesium sterate. Each subject received ebselen at one of three dosages (either 200 mg, 400 mg, or 600 mg) or received a placebo, delivered orally twice daily for four days, beginning two days prior to exposure to a controlled sound and continuing on the day of and on the day following the exposure to the controlled sound. The study was randomized to assign each subject to a treatment or placebo group. All subjects took 3 capsules in the morning (AM) and 3 capsules in the evening (PM) (each capsule either contained 200 mg ebselen or placebo), ideally 12 hours apart.

Side effects of the ebselen delivery were analyzed. Clinical signs and symptoms from physical exam were evaluated, such as shortness of breath, wheezing, coughing, hemoptysis, resting pulse oximetry, chest X-rays, and laboratory findings such as hematology, chemistry, and urinalysis. Exposure to ebselen was quantified from plasma by LC-MS/MS. Female subjects received pregnancy testing at screening, and any pregnant subjects were excluded from the study.

On Day 3 of the ebselen treatment, subjects underwent audiometric testing immediately prior to sound-exposure to establish their baseline hearing levels. Subjects listened to a 100 dBA SPL sound exposure delivered via insert earphones from a Personal Music Player (PMP, specifically an iPod®) for 4 continuous hours. The sound exposure method was similar to that discussed in Le Prell, C. et al., Ear & Hearing 33:e44-e58, 2012, hereby incorporated by reference in its entirety. The music levels were measured using Bruel & Kjaer type 4157 artificial ear couplers. Subjects listened to sound from music playlists with a choice of rock or pop music. No changes in volume or duration of the sound exposure were permitted. At 15 minutes, 75 minutes, 135 minutes, 195 minutes, 24 hours, and 1 week after sound exposure, subjects were tested for pure tone audiometric thresholds. Serial pure tone audiometry determined the incidence (greater or equal to 10 dB HL), severity (dB HL) and duration (up to 1 week post sound exposure) of the TTS. Audiometric data was collected at 250, 500, 1000, 2000, 3000, 4000, 6000, and 8000 Hz.

To maximize test validity and reliability, ASHA Guidelines for Manual Pure Tone Threshold Audiometry (2005) were followed. Audiological evaluations took place in a sound-treated testing facility having ambient noise levels conforming to ANSI S3.1-1999 standards. Pure tone audiometric testing was performed using a calibrated audiometer, capable of producing tonal stimuli, with insert earphones calibrated to meet ANSI 3.6-2004 specifications for clinical audiometers. Air conduction thresholds were conducted using a modified Hughson-Westlake procedure for test frequencies of 0.25, 0.5, 1, 2, 3, 4, 6, and 8 kHz, as described by Le Prell et al (2012). Bone conduction thresholds were conducted for test frequencies 0.25, 0.5, 1, 2, and 4 kH if the air-conduction threshold at that frequency was >15 dB and up to 25 dB HL. Furthermore, tympanometry was performed on a standard, calibrated, middle ear analyzer as a method of assessing middle ear function.

The audiometric data was collected and analyzed using a Mixed-effects Model of Repeated Measures (MMRM) to determine significant differences between treatment, ear, frequency and time. This analysis was performed to determine if the primary and secondary efficacy endpoints were achieved. Additional analyses were performed based on the incidence and duration of the TTS. Comparisons of the incidence of a significant TTS were made between an SPI-1005 treatment group and placebo based on Fisher's exact test. Comparisons of the duration or return to baseline hearing were made between a SPI-1005 treatment group and placebo using the Wilcoxon test and Bonferroni's adjustment to control for multiplicity.

The oral administration of ebselen showed a significant reduction in TTS incidence (60% vs. 20%, p<0.02), severity (4.1 vs. 1.3 dBHL at 4 kHz, p<0.001), and duration (24 vs. 1.3 hrs, p<0.01). Ebselen treatment was well-tolerated with no drug-related adverse events (CBC, Chemistry-20, Chest X-ray). The primary endpoint was a clinically relevant reduction in the severity of TTS (>50%) measured in dB HL at 4 kHz. The secondary endpoints were a clinically relevant reduction in the severity of the TTS in dB HL averaged across all frequencies tested, or across 3, 4, and 6 kHz or across 4, 6, and 8 kHz in the same ear. At 15 minutes post sound exposure, the TTS at 4 kHz was significantly reduced from 4.1 dB HL in the placebo group to 1.3 dB HL in an ebselen treatment group (p<0.003), with the 400 mg dose being the most effective based on Intent to Treat analysis (ITT). Additionally, the TTS at 4 kHz was significantly reduced at 1.25 (p<0.001), 2.25 (p<0.002), and 3.25 (p<0.003) hours post sound exposure in the 400 mg dose group vs. placebo, based on ITT. At 15 minutes post sound exposure, clinically relevant reductions in the TTS averaged across all tested frequencies (p<0.001), or the mean across 3, 4, and 6 kHz (p<0.001) or 4, 6, and 8 kHz (p<0.002) were also observed, with the 400 mg dose being the most effective based on ITT. Thus, this randomized, placebo-controlled, double blind, Phase 2 clinical trial met both primary and secondary endpoints.

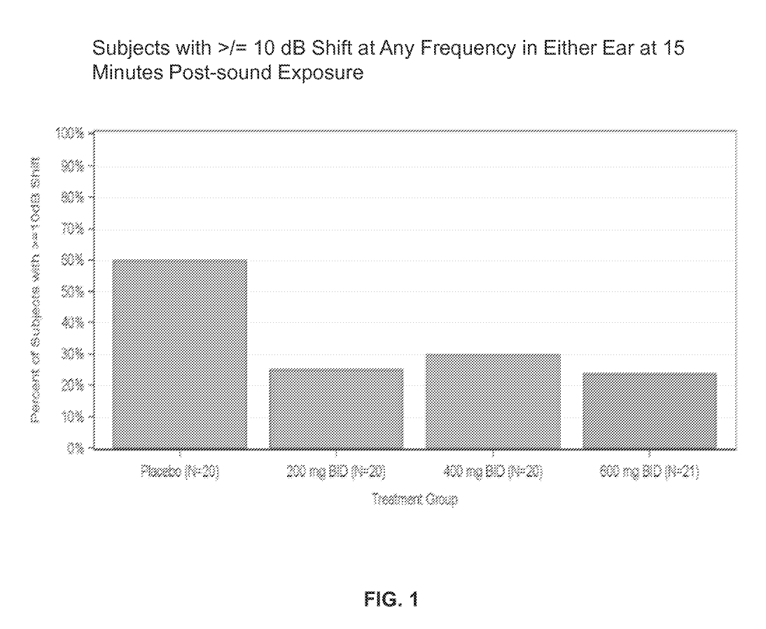

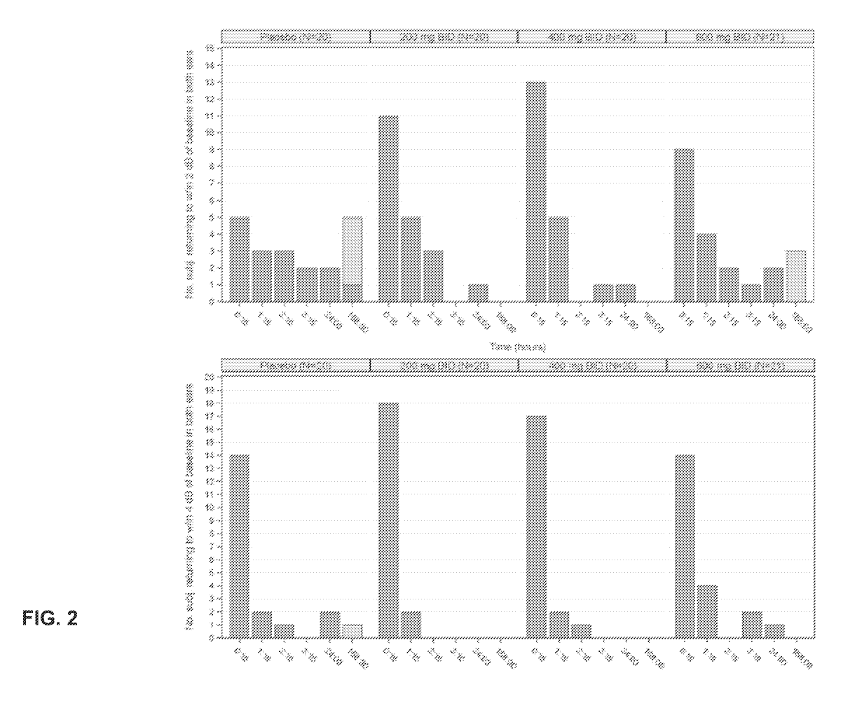

Post-hoc analyses focused on the incidence of subjects that developed a significant TTS (greater or equal to 10 dB HL in either ear) and the incidence of subjects that did not return to their baseline threshold (less than or equal to 2 dB in both ears) in less than 24 hours after sound exposure. Comparisons were made between an ebselen treatment group and the placebo group based on chi-square analysis. FIG. 1 shows that at 15 minutes post sound exposure, a clinically relevant reduction in TTS incidence from 60% in the placebo group to 20% in an ebselen treatment group was observed, with the 600 mg dose (p<0.02) being the most effective based on ITT (n=81). Significance was determined by the Exact Cochran Armitage Trend Test for each time point, and found to be highly significant at p=0.0188. This illustrates the sensitivity of the study results were accurately achieved within a relatively short period of time. A separate Fisher's exact test was performed for placebo vs. an ebselen treatment group: 200 mg (p=0.0268); 400 mg (p=0.0555); 600 mg p=0.0203). For most subjects, the return to baseline hearing occurred within 24 hours regardless of treatment. FIG. 2 shows that all ebselen treatment groups had a greater incidence of subjects that returned to baseline hearing within 24 hours compared with the placebo groups, illustrating the clinical relevance of the study.

### Example 2

### Use of a Speech Discrimination Test (SDT) with a Calibrated Sound Challenge

A speech discrimination test (SDT) was employed to determine if the calibrated sound challenge would alter a subject's ability to discrimate words. The Words in Noise Test (WINT) was found to be a clinically relevant and validated SDT in both young adults with normal hearing and in older adults with noise-induced hearing loss and age-related hearing loss. However, the WINT had not been tested before and after an acute noise exposure that induced a temporary threshold shift (TTS).

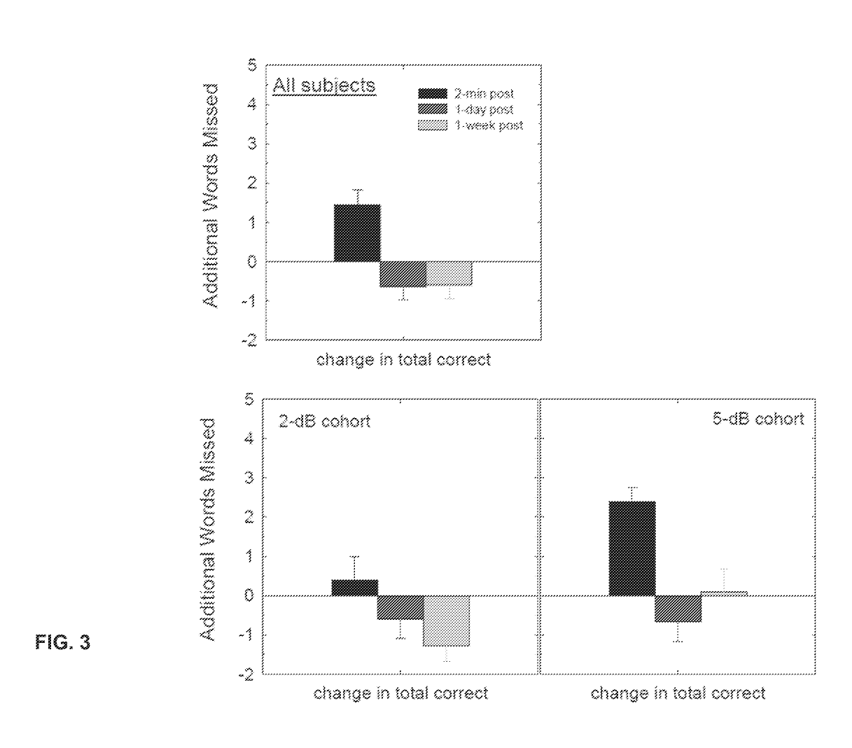

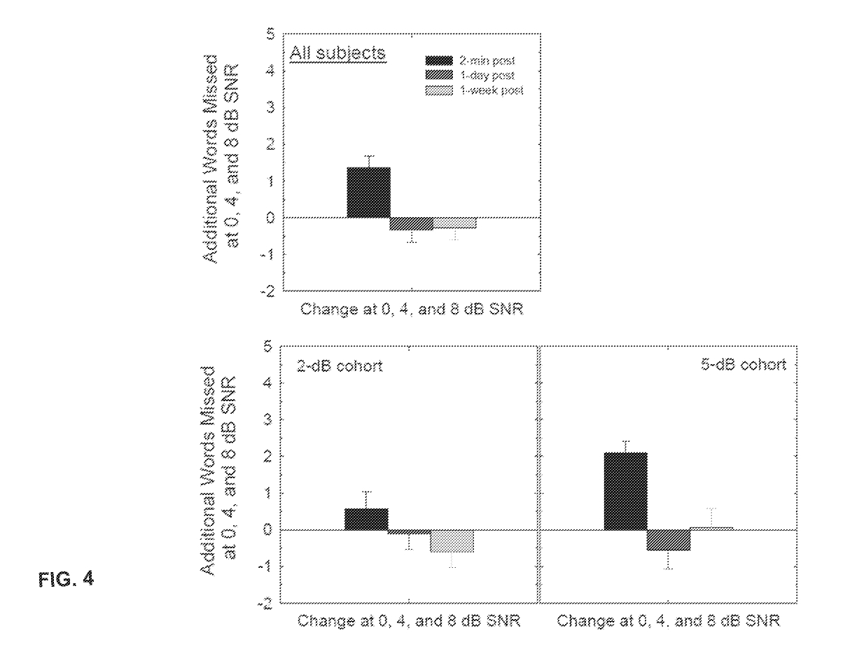

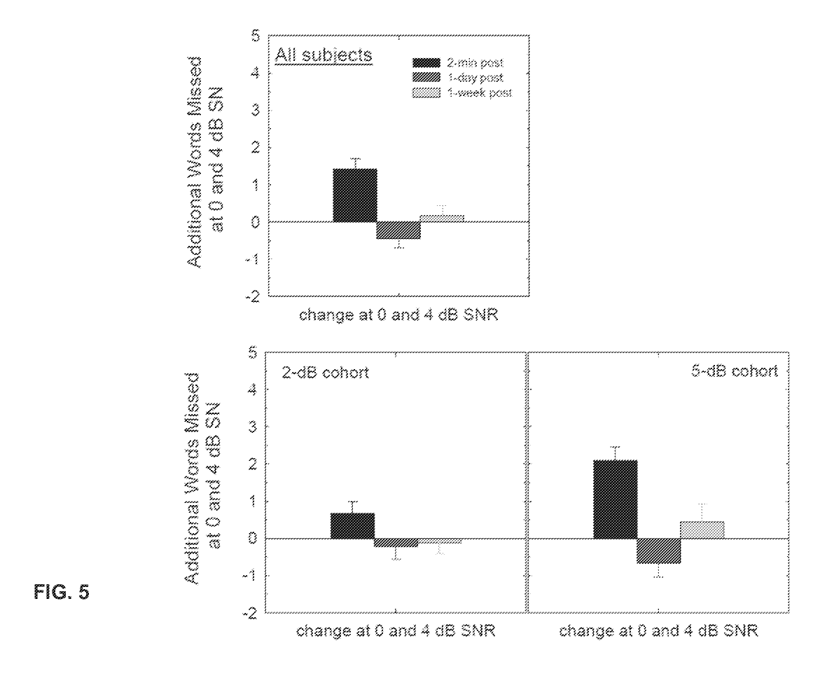

The WINT involves the presentation of 35 monosyllabic words at a constant level to each ear in background noise that is varied. The WINT is conducted at several different signal to noise ratios (SNRs), such as 24, 20, 16, 12, 8, 4 and 0. Each SNR involves the presentation of 5 different unique words to each ear. The WINT was established at baseline, and repeated at 2 min, 1 day, and 1 week post noise in test subjects. The outcome of these tests are shown in FIGS. 3, 4, and 5 as the number of additional words missed. As shown by the data in these figures, the number of words missed is highest 2 minutes post noise and then decreases 1 day and 1 week post noise. These results indicate the utility of this test for the practice of the methods disclosed herein.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A method for identifying a composition useful for the prevention or treatment of noise-induced hearing loss, the method comprising:
exposing a mammalian test subject to a candidate composition;
exposing the test subject to a calibrated sound or a noise challenge;
monitoring a temporary threshold shift (TTS) in the test subject over a period of time after exposing the test subject to the calibrated sound or noise challenge;
comparing the monitored TTS with a TTS of a control subject exposed to a control composition and a calibrated sound or a noise challenge equivalent to the calibrated sound or noise challenged exposed to the test subject; and
determining the presence or absence of a clinically relevant and statistically significant difference between the monitored TTS in the test subject and the TTS of the control subject wherein the presence of a clinically relevant and statistically significant difference identities the candidate composition as useful for prevention or treatment of noise-induced hearing loss.

2. The method of claim 1, wherein the exposing of the test subject to the candidate composition occurs prior to the exposing of the test subject to the calibrated sound or noise challenge.

3. The method of claim 1, wherein the exposing of the test subject to the candidate composition occurs after to the exposing of the test subject to the calibrated sound or noise challenge.

4. The method of claim 1, wherein the test subject is a human.

5. The method of claim 1, wherein the test subject is a mouse, rat, guinea pig, chinchilla, or monkey.

6. The method of claim 1, wherein the candidate composition is administered orally.

7. The method of claim 1, wherein the candidate composition is administeredtransdermally or transnasally.

8. The method of claim 1, wherein the candidate composition is selected from the group comprising: a glutathione peroxidase mimic, a xanthine oxidase inhibitor, a glutathione, and a glutathione precursor.

9. The method of claim 1, wherein the candidate composition is a glutathione peroxidase mimic.

10. The method of claim 1, wherein the candidate composition is selected from the group comprising: methionine, N-acetyl-DL-methionine, S-adenosylmethionine, cysteine, homocysteine, N-acetylcysteine, glutathione, glutathione ethylester, glutathione diethylester, glutathione triethylester, cysteamine, cystathione, N,N′-diacetyl-L-cystine (DiNAC), 2(R,S)-D-ribo-(1′,2′,3′,4′-tetrahydroxybutyl)-thiazolidine (RibCyst), 2-oxo-L-thiazolidine-4-carboxylic acid (OTCA), allopurinol 1-methylallopurinol, 2-methylallopurinol, methylallopurinol, 7-methylallopurinol, 1,5-dimethylallopurinol, 2,2-dimethylallopurinol, 1,7-dimethylallopurinol, 2,7-dimethylallopurinol, 5,7-dimethylallopurinol, 2,5,7-trimethylallopurinol, 1-ethyoxycarbonylallopurinol, 1-ethoxycarbonyl-5-methylallopurinol, 2-phenyl-1,2-benzoisoselenaol-3(2H)-one (ebselen), 6A,6B-diseleninic acid-6A′,6B′-selenium bridged β-cyclodextrin (6-diSeCD), and 2,2′-diseleno-bis-β-cyclodextrin (2-diSeCD).

11. The method of claim 1, wherein the test subject is exposed to the calibrated sound or noise challenge for a duration of time between 1 minute and 10 hours.

12. The method of claim 1, wherein the test subject is exposed to the calibrated sound or noise challenge for a duration of time between 0.5 hours and 4 hours.

13. The method of claim 1, wherein the test subject is exposed to the calibrated sound or noise challenge for 4 hours.

14. The method of claim 1, wherein the test subject is exposed to the calibrated sound or noise challenge at 80-110 dBA SPL.

15. The method of claim 1, wherein the test subject is exposed to the calibrated sound or noise challenge at 85-100 dBA SPL.

16. The method of claim 1, wherein the TTS is monitored by serial pure tone audiometry, auditory brainstem responses, distortion product otoacoustic emissions, or a speech discrimination test.

17. The method of claim 16, wherein the TTS is monitored by serial pure tone audiometry, and the serial pure tone audiometry determines an incidence, severity, or duration of the monitored TTS.

18. The method of claim 16, wherein the speech discrimination test is a Words in Noise Test (WINT).

19. The method of claim 1, wherein the period of time is between 2 minutes and 7 days.

20. The method of claim 1, wherein the period of time is between 15 minutes and 24 hours.

21. The method of claim 1, wherein the test subject has normal hearing or slight hearing loss.

22. The method of claim 1, wherein the statistically significant difference is a 20%-60% difference between an incidence of the monitored TTS and an incidence of the TTS of the control subject.

23. The method of claim 1, wherein the candidate composition is administered to the test subject at a dosage between 100 mg and 2000 mg.

24. The method of claim 1, wherein the candidate composition is administered to the test subject at a dosage of 200 mg, 400 mg, or 600 mg.

25. The method of claim 1, wherein a clinically relevant difference comprises a 10 dB threshold shift.

26. The method of claim 1, wherein a clinically relevant difference comprises a return to baseline hearing within 24 hours of the noise-induced hearing loss.

27. The method of claim 1, wherein determining the presence or absence of a clinically relevant and statistically significant difference comprises comparing an incidence of a significant TTS between the monitored TTS in the test subject and the TTS of the control subject.

28. The method of claim 1, wherein determining the presence or absence of a clinically relevant and statistically significant difference comprises comparing a time to return to baseline hearing between the monitored TTS in the test subject and the TTS of the control subject.

29. The method of claim 1, wherein determining the presence or absence of a clinically relevant and statistically significant difference comprises a pairwise comparison between the monitored TTS in the test subject and the TTS of the control subject, based on a mixed-effects multiple repeated measures of analysis.

30. The method of claim 1, wherein the candidate composition is administered to the test subject twice daily for four days.

31. The method of claim 30, wherein a first administration of the candidate composition occurs two days before exposing the test subject to the calibrated sound or noise challenge.

32. The method of claim 1, wherein the candidate composition is co-administered to the test subject combined with a xanthine oxidase inhibitor, a glutathione, or a glutathione precursor.

33. The method of claim 1, wherein the test subject and the control subject are the same subject.

34. The method of claim 1, wherein the test subject comprises a population of test subjects and the control subject comprises a population of control subjects

35. The method of claim 1 or claim 34, wherein the test subject and the control subject are different subjects.

36. The method of claim 1, wherein the test subject is between 18 and 31 years in age.

37. The method of claim 1, wherein the test subject had passed a pure tone audiometry test prior to exposing the subject to the candidate composition, and the calibrated sound or noise challenge.

38. A method for identifying a subject at risk for noise-induced hearing loss, the method comprising:
exposing a mammalian test subject to a calibrated sound or a noise challenge;
monitoring a temporary threshold shift (TTS) in the test subject over a period of time after exposing the test subject to the calibrated sound or noise challenge;
comparing the monitored TTS with a control TTS; and
determining the presence or absence of a clinically relevant and statistically significant difference between the monitored TTS in the test subject and the control TTS, wherein the presence of a statistically significant difference identifies the test subject as being at risk for noise-induced hearing loss.

39. The method of claim 38, wherein the test subject is exposed to the calibrated sound or noise challenge for a duration of time between 1 minute and 10 hours.

40. The method of claim 38, wherein the test subject is exposed to the calibrated sound or noise challenge for a duration of time between 0.5 hours and 4 hours.

41. The method of claim 38, wherein the test subject is exposed to the calibrated sound or noise challenge for 4 hours.

42. The method of claim 38, wherein the test subject is exposed to the calibrated sound or noise challenge at 80-110 dBA SPL.

43. The method of claim 1, wherein the test subject is exposed to the calibrated sound or noise challenge at 85-100 dBA SPL.

44. The method of claim 38, wherein the TTS is monitored by serial pure tone audiometry, auditory brainstem responses, distortion product otoacoustic emissions, or a speech discrimination test.

45. The method of claim 44, wherein the TTS is monitored by serial pure tone audiometry, and the serial pure tone audiometry determines an incidence, severity, or duration of the monitored TTS.

46. The method of claim 44, wherein the speech discrimination test is a Words in Noise Test (WINT).

47. The method of claim 38, wherein the period of time is between 2 minutes and 7 days.

48. The method of claim 38, wherein the period of time is between 15 minutes and 24 hours.

49. The method of claim 38, wherein the test subject has normal hearing or slight hearing loss.

50. The method of claim 38, wherein the statistically significant difference is a 20%-60% difference between an incidence of the monitored TTS and an incidence of the TTS of the control subject.

51. The method of claim 38, wherein a clinically relevant difference comprises a 10 dB threshold shift.

52. The method of claim 38, wherein a clinically relevant difference comprises a return to baseline hearing within 24 hours of the noise-induced hearing loss.

53. The method of claim 38, wherein determining the presence or absence of a clinically relevant and statistically significant difference comprises comparing an incidence of a significant TTS between the monitored TTS in the test subject and the TTS of the control subject.

54. The method of claim 38, wherein determining the presence or absence of a clinically relevant and statistically significant difference comprises comparing a time to return to baseline hearing between the monitored TTS in the test subject and the TTS of the control subject.

55. The method of claim 38, wherein determining the presence or absence of a clinically relevant and statistically significant difference comprises a pairwise comparison between the monitored TTS in the test subject and the TTS of the control subject, based on a mixed-effects multiple repeated measures of analysis.

56. The method of claim 38, wherein the test subject is between 18 and 31 years in age.
